(19) United States
(12) Patent Application Publication
(10) Pub. No.: US 2018/0243325 A1
(43) Pub. Date: Aug. 30, 2018

(54) METHOD FOR INHIBITING ABSORPTION OF AND/OR PROMOTING EXCRETION OF LIPIDS USING D-PSICOSE

(71) Applicant: CJ CHEILJEDANG CORPORATION, Seoul (KR)

(72) Inventors: Myung-Sook CHOI, Daegu (KR); Eun-young KWON, Gyeonggi-do (KR); Youngji HAN, Chilgok-gun (KR)

(21) Appl. No.: 15/756,919

(22) PCT Filed: Sep. 1, 2016
(86) PCT No.: PCT/KR2016/009812

§ 371 Date: Mar. 1, 2018

(30) Foreign Application Priority Data
Sep. 1, 2015 (KR) 10-2015-0123437

(51) Int. Cl. A61K 31/7004; A61P 3/06

(52) U.S. Cl. CPC A61K 31/7004; A61P 3/06

(57) ABSTRACT

The present invention relates to a composition for preventing or treating lipid-related metabolic diseases. The composition according to the present invention comprises D-psicose as an active ingredient. D-psicose has the ability to inhibit lipid absorption in the small intestine and considerably increase lipid levels in feces. Due to its ability, D-psicose is effective in inhibiting the formation of body fat. In addition, D-psicose reduces body weight, body fat mass, and plasma lipid to normal levels to normalize body weight, body fat mass, and plasma lipid profiles. Due to these advantages, D-psicose is expected to find application in pharmaceutical drugs, health functional foods, and functional foods suitable for preventing and/or treating lipid-related metabolic diseases.

### CROSS-REFERENCE TO RELATED APPLICATIONS

The present specification is a U.S. National Stage of International Patent Application No. PCT/KR2016/009812 filed Sep. 1, 2016, which claims priority to and the benefit of Korean Patent Application No. 10-2015-0123437 filed in the Korean Intellectual Property Office on Sep. 1, 2015, the entire contents of which are incorporated herein by reference.

### TECHNICAL FIELD

The present invention relates to a method for inhibiting lipid absorption and/or promoting lipid excretion using D-psicose.

### BACKGROUND ART

D-psicose, the C-3 epimer of D-fructose, is a natural sugar present in a trace amount in commercial mixtures of D-glucose and D-fructose obtained from hydrolysis of sucrose or isomerization of D-glucose. D-psicose is a monosaccharide with a sweetness of 70% relative to sugar. D-psicose was reported to be a sweetener that contains few or no calories because it is not metabolized in a body and that has little effect on body weight gain because it functions to inhibit the formation of body fat. According to a recently published report, D-psicose has non-cariogenic and anti-cariogenic effects. Under these circumstances, D-psicose is currently under active development as a sweetener that has the potential to replace sugar while assisting in dental health. Thus, D-psicose has received attention as a sweetener for preventing weight gain in the food industry due to characteristics and functionalities thereof.

D-psicose is generally recognized as safe (GRAS) by the United States Department of Agriculture (USDA). Some studies reported that D-psicose affects lipid metabolism (Yasuo nagata et al., J. Agric, Food Chem. 2015, 63, 3168-3176), but, to the best of our knowledge, no report on the use of D-psicose for reducing lipid absorption and promoting lipid excretion has been published to date.

The present inventors have found the fact that D-psicose has functions of inhibiting lipid absorption in the small intestine and considerably increasing lipid levels in feces, reduces body weight, body fat mass, and plasma levels of lipids (including free fatty acids, triglycerides, total cholesterol, non-high-density lipoprotein cholesterol, and apolipoprotein B) such that body weight, body fat mass, and plasma lipid profiles are normalized, and is effective in reducing the activity of fatty acid synthase (FAS). The present invention has been accomplished based on this finding.

### DISCLOSURE

### Technical Problem

It is an aspect of the present invention to provide a method for inhibiting the absorption of lipids ingested by a subject and/or promoting the excretion of the ingested lipids comprising administering D-psicose to the subject, use of a composition comprising D-psicose for reducing the absorption of food lipids, inhibitors of a fatty acid synthase (FAS) comprising D-psicose, a method for inhibiting the activity of fatty acid synthase in a subject comprising administering D-psicose to the subject, a method for preventing, ameliorating or treating hyperlipidemia, arteriosclerosis or fatty liver comprising administering a pharmaceutically effective amount of D-psicose to a subject in need of thereof, and a composition for preventing, ameliorating or treating hyperlipidemia, arteriosclerosis or fatty liver comprising D-psicose.

The present invention will be now described in more detail. Disclosures that are not included herein will be readily recognized and appreciated by those skilled in the art, and thus a description thereof is omitted.

### Technical Solution

One aspect of the present invention provides a method for inhibiting the absorption of lipids ingested by a subject and/or promoting the excretion of the ingested lipids, comprising administering D-psicose to the subject.

The subject includes mammals including humans and non-human mammals. Examples of the non-human mammals include, but are not limited to, mice, rats, dogs, cats, horses, cows, sheep, goats, pigs, and rabbits.

The lipids include animal lipids and vegetable lipids but are not limited thereto. Specifically, the lipids may be animal lipids, vegetable lipids or combinations thereof. More specifically, the lipids may be ones that are present in food or feed.

The administration may be oral administration or parenteral administration (e.g., intravenous administration, subcutaneous administration, intraperitoneal administration or topical application). Specifically, the administration may be oral administration.

D-psicose may be administered in an amount of 10 to 50 parts by weight, relative to 100 parts by weight of the lipids ingested by the subject. More specifically, D-psicose may be administered in an amount of 10-40, 10-30, 10-25, 15-50, 15-40, 15-30, 15-25, 20-50, 20-40, 20-30, 20-25 or 25 parts by weight, relative to 100 parts by weight of the lipids ingested by the subject.

In one embodiment of the present invention, the absorption may be absorption in the small intestine. Specifically, when administered to high-fat diet-fed obese mice, D-psicose reduces mRNA expression of genes (CD36, FATP4, and ApoB48) involved in lipid absorption in the small intestine. Specifically, D-psicose reduces mRNA expression of genes (ABCGS and ABCG8) involved in lipid excretion.

A further aspect of the present invention provides use of a composition comprising D-psicose for inhibiting the absorption of food lipids and/or promoting the excretion of food lipids.

To the best of our knowledge, this is the first report on the mechanism by which D-psicose, which is widely used as a sweetener, inhibits the absorption of food lipids and promotes the excretion of food lipids. Details of the mechanism are the same as those described above.

Yet another aspect of the present invention provides a method for inhibiting the activity of fatty acid synthase (FAS) in a subject comprising administering a fatty acid synthase inhibitor comprising D-psicose or D-psicose to the subject.

In one embodiment of the present invention, D-psicose reduces fatty acid β-oxidation activity in the liver. Alternatively, D-psicose may induce fatty acid β-oxidation activity in adipose tissue.

Yet another aspect of the present invention provides a method for preventing, ameliorating or treating hyperlipidemia, arteriosclerosis or fatty liver comprising administering a pharmaceutically effective amount of D-psicose to a subject in need of such prevention, amelioration or treatment.

Yet another aspect of the present invention provides a composition for preventing, ameliorating or treating hyperlipidemia, arteriosclerosis or fatty liver comprising D-psicose.

As used herein, the term “hyperlipidemia” refers to a disease caused by abnormally high blood fat levels as a result of insufficient metabolism of fats such as triglycerides and cholesterol. More specifically, hyperlipidemia is characterized by increased levels of lipids (including triglycerides, LDL cholesterol, phospholipids, and free fatty acids) in the blood, and hyperlipidemia is including hypercholesterinemia or hypertriglyceridemia, which occurs frequently from increased levels of lipids.

As used herein, the term “arteriosclerosis” refers to a disease where cholesterol is deposited on the inner walls of the arteries or vascular endothelial cells proliferate to narrow or occlude the arteries, causing poor blood circulation to the peripheries.

As used herein, the term “fatty liver” refers to a condition where fat accumulates excessively in hepatic cells due to the disorder of fat metabolism in the liver. Fatty liver is a cause of various diseases such as angina, myocardial infarction, stroke, arteriosclerosis, fatty liver and pancreatitis.

As used herein, the term “prevention” or “preventing” means all actions that inhibit or delay the development of target diseases. Specifically, this term means administering D-psicose to inhibit or delay the development of hyperlipidemia, arteriosclerosis, and fatty liver symptoms (for example, elevated plasma free fatty acid, triglyceride, total cholesterol, non-HDL cholesterol, and Apo B levels, high arteriosclerosis index (AI), increased fatty acid, triglyceride, and cholesterol levels in hepatic tissue, and increased size of adipocytes).

As used herein, the term “amelioration” or “ameliorating” means all actions that alleviate or relieve symptoms and side effects of diseases. As used herein, the term “treatment” or “treating” refers to all actions that alleviate or beneficially change symptoms and side effects of diseases. Specifically, these terms mean administering D-psicose to alleviate, palliate or relieve hyperlipidemia, arteriosclerosis or fatty liver symptoms, resulting in reduced plasma free fatty acid, triglyceride, total cholesterol, non-HDL cholesterol, or Apo B level, low arteriosclerosis index (AI), reduced fatty acid, triglyceride or cholesterol level or reduced size of adipocytes in hepatic tissue.

As demonstrated in Examples section that follows, it was found that D-psicose significantly reduces free fatty acids, triglyceride, total cholesterol, non-HDL cholesterol, Apo B, leptin, resistin levels and leptin/adiponectin ratio in the plasma of high-fat diet-induced obese mice such that the levels and ratio are maintained similarly to those in the normal diet group, increases the levels of plasma HDL-cholesterol and Apo A-1 to higher values than those in the normal diet group, and lowers arteriosclerosis index (AI), thus being effective in preventing, ameliorating or treating hyperlipidemia or arteriosclerosis. It was also found that D-psicose reduces the activity of fatty acid synthase (FAS), the levels of fatty acids, triglycerides, cholesterol, and the size of adipocytes in the liver tissues of high-fat diet-induced obese mice to inhibit the development of fatty liver by high-fat diet. Furthermore, D-psicose was confirmed to reduce mRNA expression of genes involved in fatty acid synthesis in the livers of high-fat diet-induced obese mice, thus being effective in preventing or treating fatty liver.

Based on these findings, it can be concluded that D-psicose has functions of inhibiting lipid absorption in the small intestine and considerably increasing lipid levels in feces and is effective in reducing plasma lipid level to normalize plasma lipid profiles. Therefore, D-psicose can find application in pharmaceutical drugs and foods (specifically, health functional foods) for preventing, ameliorating or treating hyperlipidemia, arteriosclerosis or fatty liver.

The composition according to the present invention can be administered orally or parenterally (e.g., intravenously, subcutaneously, intraperitoneally or topically) depending on the intended use. Specifically, the composition according to the present invention can be administered orally.

The composition according to the present invention may be used as a pharmaceutical composition. In this case, the composition according to the present invention may further comprise at least one pharmaceutically acceptable carrier suitable for administration. The pharmaceutically acceptable carrier may be used in admixture with one or more components selected from saline solution, sterilized water, Ringer's solution, buffered saline, dextrose solution, maltodextrin solution, glycerol, and ethanol. One or more general additives, such as antioxidants, buffer solutions, and bacteriostatic agents may be added, if needed. The composition according to the present invention may be prepared into injectable formulations (such as aqueous solutions, suspensions or emulsions), pills, capsules, granules, or tablets. In this case, the composition according to the present invention may further comprise one or more additives selected from diluents, dispersants, surfactants, binders, and lubricants. The composition according to the present invention may be prepared into various formulations depending on the type of disease or the kind of components according to any suitable method known in the art or any of the conventional procedures disclosed in Remington's Pharmaceutical Science (the newest edition), Mack Publishing Company, Easton Pa.

The dose of the pharmaceutical composition according to the present invention may be determined taking into consideration various factors, including body weight, age, sex, health condition, diet, time and mode of administration, and rate of excretion, and severity of disease. A daily dose of D-psicose may be range from about 0.0001 to about 600 mg/kg, preferably about 0.001 to about 500 mg/kg, and may be administered in single or divided doses per day.

The pharmaceutical composition according to the present invention may be used alone or in combination with surgical operation, hormone therapy, drug treatment, and biological regulators.

The composition according to the present invention may be used as a food or health food composition. In this case, D-psicose may be added as it is or in combination with other foods or food ingredients and may be suitably used according to any general method known in the art. The amount of the active ingredient can be determined according to the purpose of use (prevention, health or therapeutic regimen). The food composition may be used without limitation in any food or health food that includes lipids. Examples of suitable foods include meats, sausages, breads, cakes, chocolates, candies, snacks, crackers, cookies, pizza, flour products (e.g., instant noodles), gums, dairy products (including ice creams), soups, ketchups, sauces, gravies, dressings, beverages, teas, drinks, alcoholic drinks, and vitamin complexes.

The food or health food composition according to the present invention may further comprise various flavors or natural carbohydrates, like general beverages. The natural carbohydrates include monosaccharides such as glucose and fructose, disaccharides such as maltose and sucrose, polysaccharides such as dextrin and cyclodextrin, and sugar alcohols such as xylitol, sorbitol, and erythritol. The food or health food composition according to the present invention may further comprise natural or synthetic sweetening agents. The natural sweetening agents include thaumatin and stevia extracts. The synthetic sweetening agents include saccharin and aspartame. The natural carbohydrate is typically used in an amount of about 0.01 to about 0.20 g, specifically 0.04 to 0.10 g, per 100 ml of the food or health food composition.

The food or health food composition according to the present invention may further comprise a variety of nutrients, vitamins, electrolytes, flavors, coloring agents, pectic acid and its salts, alginic acid and its salts, organic acids, protective colloidal thickeners, pH adjusting agents, stabilizers, preservatives, glycerin, alcohols, and carbonating agent for carbonated beverages. The food or health food composition according to the present invention may further comprise fruit flesh for the production of natural fruit juices, fruit juice beverages and/or vegetable beverages. These ingredients may be used independently or in combination. The total amount of the ingredients added may be in the range of 0.01 to 0.20 parts by weight, relative to 100 parts by weight of the food or health food composition.

The use of reducing the absorption of food lipids, the inhibitor of fatty acid synthase activity, the method for inhibiting the activity of fatty acid synthase, the method for preventing, ameliorating or treating hyperlipidemia, arteriosclerosis or fatty liver, and the composition according to the present invention share D-psicose, lipid, administration, and subject in common with the method for inhibiting lipid absorption and/or promoting lipid absorption, and a description thereof is thus omitted to avoid excessive complexity of the specification.

### Advantageous Effects

The present inventors have attempted to clarify the physiological activity of D-psicose by assigning isocaloric diets to each diet group to exclude the effect of D-psicose on calorie reduction, and as a result, found that D-psicose has functions of inhibiting lipid absorption in the small intestine and considerably increasing lipid levels in feces to inhibit fat production and reduces body weight, body fat mass, and plasma lipid levels such that body weight, body fat mass, and plasma lipid profiles are normalized in a short time. Due to these advantages, it is expected that D-psicose will be used to prevent and/or treat lipid-related metabolic diseases.

### DESCRIPTION OF DRAWINGS

FIG. 1 shows changes in the body weight of C57BL/6J mice fed both D-psicose and high-fat diet for 16 weeks [normal diet group (ND), high-fat diet group (HFD), PSI group (HFD+5% D-psicose, w/w), ERY group (HFD+5% erythritol, w/w), GLU group (HFD+5% D-glucose, w/w), and FRU group (HFD+5% D-fructose, w/w)].

FIGS. 2A and 2B show changes in plasma triglyceride and total cholesterol levels in C57BL/6J mice fed both D-psicose and high-fat diet for 16 weeks [normal diet group (ND), high-fat diet group (HFD), PSI group (HFD+5% D-psicose, w/w), ERY group (HFD+5% erythritol, w/w), GLU group (HFD+5% D-glucose, w/w), and FRU group (HFD+5% D-fructose, w/w)].

FIG. 3 shows the influences of D-psicose on plasma leptin, resistin, and adiponectin levels, and leptin:adiponectin ratio (L:A ratio) in high-fat diet-induced obese mice [normal diet group (ND), high-fat diet group (HFD), PSI group (HFD+5% D-psicose, w/w), ERY group (HFD+5% erythritol, w/w), GLU group (HFD+5% D-glucose, w/w), and FRU group (HFD+5% D-fructose, w/w)].

FIGS. 4A-4C show the influences of D-psicose on (A) hepatic lipid profiles, (B) hepatic lipid regulating enzyme activities, and (C) hepatic tissue morphologies of high-fat diet-induced obese mice [normal diet group (ND), high-fat diet group (HFD), PSI group (HFD+5% D-psicose, w/w), ERY group (HFD+5% erythritol, w/w), GLU group (HFD+5% D-glucose, w/w), and FRU group (HFD+5% D-fructose, w/w)].

FIGS. 5A and 5B show the influences of D-psicose on (A) hepatic lipid regulating enzyme activities and (B) hepatic tissue morphologies of high-fat diet-induced obese mice [normal diet group (ND), high-fat diet group (HFD), PSI group (HFD+5% D-psicose, w/w), ERY group (HFD+5% erythritol, w/w), GLU group (HFD+5% D-glucose, w/w), and FRU group (HFD+5% D-fructose, w/w)].

FIG. 6 shows the influences of D-psicose on mRNA expression of genes (FAS, ACC1, CPT1α, and CPT2) involved in fatty acid synthesis and oxidation in the livers of high-fat diet-induced obese mice [normal diet group (ND), high-fat diet group (HFD), PSI group (HFD+5% D-psicose, w/w), ERY group (HFD+5% erythritol, w/w), GLU group (HFD+5% D-glucose, w/w), and FRU group (HFD+5% D-fructose, w/w)].

FIG. 7 shows the influences of D-psicose on lipid levels in feces from high-fat diet-induced obese mice [normal diet group (ND), high-fat diet group (HFD), PSI group (HFD+5% D-psicose, w/w), ERY group (HFD+5% erythritol, w/w), GLU group (HFD+5% D-glucose, w/w), and FRU group (HFD+5% D-fructose, w/w)].

FIG. 8 shows the influences of D-psicose on mRNA expression of genes (CD36, FATP4, ApoB48, ABCG5 and ABCG8) involved in lipid absorption and excretion in the small intestines of high-fat diet-induced obese mice [normal diet group (ND), high-fat diet group (HFD), PSI group (HFD+5% D-psicose, w/w), ERY group (HFD+5% erythritol, w/w), GLU group (HFD+5% D-glucose, w/w), and FRU group (HFD+5% D-fructose, w/w)].

FIG. 9 is a diagram schematically showing the roles of D-psicose on lipid metabolism in the small intestine, liver, and adipose tissues of high-fat diet-induced obese mice, based on the results shown in FIGS. 1 to 8.

### BEST MODE

The present invention provides a method for inhibiting lipid absorption and fatty acid synthase (FAC) activity in a subject, comprising administering D-psicose to the subject, an inhibitor of fatty acid synthase comprising D-psicose, a method for preventing or treating hyperlipidemia, arteriosclerosis or fatty liver comprising administering a pharmaceutically effective amount of D-psicose to a subject in need of thereof, and a composition for preventing or treating hyperlipidemia, arteriosclerosis or fatty liver comprising D-psicose.

The composition is intended to include a pharmaceutical composition and a food composition.

Hereinafter, preferred embodiments are presented to assist in understanding the invention. The following examples are provided for a better understanding of the invention and are not intended to limit the scope of the invention.

### Example 1: Influences of D-Psicose on Body Weight, Organ Weight, and Adipose Tissue Weight of High-Fat Diet-Induced Obese Mice

The following experiment was conducted to investigate the influences of D-psicose on the body weight, organ weight, and adipose tissue weight of high-fat diet-induced obese mice.

First, 4-week-old male C57BL/6J mice (60 total) were purchased from Jackson Laboratory. The animals were acclimated to the vivarium in a thermo-hygrostat (20-23° C., 45-65%) under a 12 h light/dark cycle and fed a pelletized commercial non-purified diet for 1 week after arrival. The mice were then randomly divided into 6 groups (n=10) and fed the respective experimental diets for 16 weeks: normal diet group (ND, American Institute of Nutrition (AIN)-76 semi-synthetic diet), high-fat diet group (HFD, 20% fat+1% cholesterol based on AIN-76 diet), PSI group (HFD+5% D-psicose, w/w, Sigma Chemical Company), ERY group (HFD+5% erythritol, w/w, Sigma Chemical Company), GLU group (HFD+5% D-glucose, w/w, Sigma Chemical Company), and FRU group (HFD+5% D-fructose, w/w, Sigma Chemical Company). All high-fat diet-fed groups were allowed to ingest the same calories by pair feeding based on the PSI group. The mice had ad libitum access to distilled water during the experimental period. Their feed intakes and body weights were measured daily and biweekly, respectively. The organ weights and the adipose tissue weights of the mice were measured after sacrificing the animals.

This animal study protocol was approved by the Ethics Committee for Animal Studies at Kyungpook National University, Korea (approval No. KNU 2013-18).

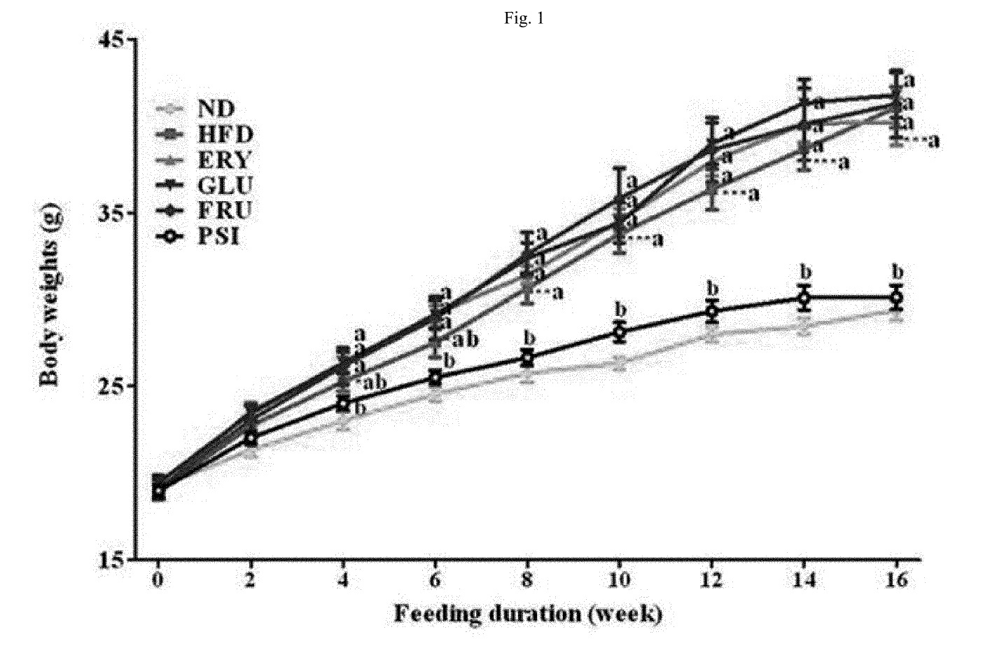

Changes in the body weight of C57BL/6J mice fed both D-psicose and high-fat diet for 16 weeks are shown in FIG. 1 and Table 2. The organ weights and adipose tissue weights of the mice are shown in Table 2.

TABLE 1
Compositions of experimental diets (% of diet, w/w)

| Ingredient (g) | ND | HFD | ERY | GLU | FRU | PSI |
|---|---|---|---|---|---|---|
| Casein | 200.00 | 200.00 | 200.00 | 200.00 | 200.00 | 200.00 |
| D,L-methionine | 3.00 | 3.00 | 3.00 | 3.00 | 3.00 | 3.00 |
| Corn starch | 150.00 | 111.00 | 111.00 | 111.00 | 111.00 | 111.00 |
| Sucrose | 500.00 | 370.00 | 320.00 | 320.00 | 320.00 | 320.00 |
| Cellulose powder | 50.00 | 50.00 | 50.00 | 50.00 | 50.00 | 50.00 |
| Corn oil | 50.00 | 30.00 | 30.00 | 30.00 | 30.00 | 30.00 |
| Lard | — | 170.00 | 170.00 | 170.00 | 170.00 | 170.00 |
| Mineral Mixture (AIN-76)* | 35.00 | 42.00 | 42.00 | 42.00 | 42.00 | 42.00 |
| Vitamin mix (AIN-76)^{†} | 10.00 | 12.00 | 12.00 | 12.00 | 12.00 | 12.00 |
| Choline bitartrate | 2.00 | 2.00 | 2.00 | 2.00 | 2.00 | 2.00 |
| Cholesterol | — | 10.00 | 10.00 | 10.00 | 10.00 | 10.00 |
| tert-Butylhydroquinone | 0.01 | 0.04 | 0.04 | 0.04 | 0.04 | 0.04 |
| D-psicose | | | | | | 50.00 |
| D-Glucose | | | | 50.00 | | |
| D-Fructose | | | | | 50.00 | |
| Erythritol | | | 50.00 | | | |
| Total (g) | 1000.0 | 1000.0 | 1000.0 | 1000.0 | 1000.0 | 1000.0 |
| Calorie (kcal/kg) | 3902 | 4584 | 4384 | 4584 | 4584 | 4384 |
| Calorie (kcal/g) | 3.902 | 4.584 | 4.384 | 4.584 | 4.584 | 4.384 |

*Mineral mixture (AIN-76): calcium phosphate 500 g/kg, NaCl 74 g/kg, potassium citrate 2220 g/kg, potassium sulfate 52 g/kg, magnesium oxide 24 g/kg, manganese carbonate 3.5 g/kg, ferric citrate 6 g/kg, zinc carbonate 1.6 g/kg, copper carbonate 0.3 g/kg, potassium iodate 0.01 g/kg, sodium selenite 0.01 g/kg, chromium potassium sulfate 0.55 g/kg, sucrose 118.03 g/kg
^{†}Vitamin mixture AIN-76: thiamine HCl 0.6 g/kg, riboflavin 0.6 g/kg, pyridoxine HCl 0.7 g/kg, niacin 3 g/kg, calcium pantothenate 1.6 g/kg, folic acid 0.2 g/kg, biotin 0.02 g/kg, vitamin B₁₂ 1 g/kg, vitamin A (500 000 IU/g) 0.8 g/kg, vitamin D3 (400 000 IU/g) 0.25 g/kg, vitamin E acetate (500 IU/g) 10 g/kg, menadione sodium bisulfate 0.04 g/kg, sucrose 981.15 g/kg.

| Ingredient (g) | ND | HFD | ERY | GLU | FRU | ALL |
|---|---|---|---|---|---|---|
| Casein | 200.00 | 200.00 | 200.00 | 200.00 | 200.00 | 200.00 |
| D,L-methionine | 3.00 | 3.00 | 3.00 | 3.00 | 3.00 | 3.00 |
| Corn starch | 150.00 | 111.00 | 111.00 | 111.00 | 111.00 | 111.00 |
| Sucrose | 500.00 | 370.00 | 320.00 | 320.00 | 320.00 | 320.00 |
| Cellulose powder | 50.00 | 50.00 | 50.00 | 50.00 | 50.00 | 50.00 |
| Corn oil | 50.00 | 30.00 | 30.00 | 30.00 | 30.00 | 30.00 |
| Lard | — | 170.00 | 170.00 | 170.00 | 170.00 | 170.00 |
| Mineral Mixture (AIN-76)* | 35.00 | 42.00 | 42.00 | 42.00 | 42.00 | 42.00 |
| Vitamin mix (AIN-76)^{†} | 10.00 | 12.00 | 12.00 | 12.00 | 12.00 | 12.00 |
| Choline bitartrate | 2.00 | 2.00 | 2.00 | 2.00 | 2.00 | 2.00 |
| Cholesterol | — | 10.00 | 10.00 | 10.00 | 10.00 | 10.00 |
| tert-Butylhydroquinone | 0.01 | 0.04 | 0.04 | 0.04 | 0.04 | 0.04 |
| D-Allulose | | | | | | 50.00 |
| D-Glucose | | | | 50.00 | | |
| D-Fructose | | | | | 50.00 | |
| Erythritol | | | 50.00 | | | |
| Total (g) | 1000.0 | 1000.0 | 1000.0 | 1000.0 | 1000.0 | 1000.0 |
| Calorie (kcal/kg) | 3902 | 4584 | 4384 | 4584 | 4584 | 4384 |
| Calorie (kcal/g) | 3.902 | 4.584 | 4.384 | 4.584 | 4.584 | 4.384 |

TABLE 2

| | ND | | HFD | | ERY | | GLU | | FRU | | PSI | |
|---|---|---|---|---|---|---|---|---|---|---|---|---|
| InitialBodyweight (g) | 19.20 ± | 0.37 | 18.97 ± | 0.36 | 19.00 ± | 0.54 | 19.45 ± | 0.42 | 19.08 ± | 0.59 | 18.99 ± | 0.35 |
| FinalBodyweight (g) | 29.37 ± | 0.58 | 39.38 ± | 1.40***^{a} | 40.22 ± | 1.36^{a} | 41.77 ± | 1.31^{a} | 41.26 ± | 1.93^{a} | 30.13 ± | 0.69^{b} |
| BWG (g/16 weeks) | 0.64 ± | 0.002 | 1.28 ± | 0.08***^{a} | 1.32 ± | 0.10^{a} | 1.39 ± | 0.07^{a} | 1.39 ± | 0.09^{a} | 0.70 ± | 0.04^{b} |
| Food Intake (g/day) | 3.12 ± | 0.10 | 3.03 ± | 0.25 | 3.17 ± | 0.26 | 3.03 ± | 0.25 | 3.03 ± | 0.25 | 3.17 ± | 0.26 |
| Energy Intake (kcal/day) | 12.16 ± | 0.39 | 13.88 ± | 1.15 | 13.88 ± | 1.15 | 13.88 ± | 1.15 | 13.88 ± | 1.15 | 13.88 ± | 1.15 |
| FER | 0.029 ± | 0.001 | 0.060 ± | 0.002***^{a} | 0.059 ± | 0.003^{a} | 0.065 ± | 0.003^{a} | 0.065 ± | 0.003^{a} | 0.031 ± | 0.002^{b} |

Organ weights (g/100 g Bodyweight)

| | | | | | | | | | | | | |
|---|---|---|---|---|---|---|---|---|---|---|---|---|
| Muscle | 1.08 ± | 0.02 | 0.85 ± | 0.03***^{b} | 0.77 ± | 0.03^{b} | 0.82 ± | 0.03^{b} | 0.83 ± | 0.04^{b} | 1.01 ± | 0.02^{a} |
| Liver | 3.41 ± | 0.05 | 5.49 ± | 0.32***^{a} | 5.37 ± | 0.25^{a} | 5.22 ± | 0.15^{a} | 5.68 ± | 0.35^{a} | 4.35 ± | 0.08^{b} |
| Kidney | 0.98 ± | 0.02 | 0.81 ± | 0.04*^{b} | 0.77 ± | 0.04^{b} | 0.74 ± | 0.02^{b} | 0.84 ± | 0.05^{b} | 1.03 ± | 0.02^{a} |

Adipose times weight (g/100 g Body weight)

| | | | | | | | | | | | | |
|---|---|---|---|---|---|---|---|---|---|---|---|---|
| Perinephric fat | 0.43 ± | 0.04 | 1.25 ± | 0.09***^{a} | 0.92 ± | 0.10^{c} | 1.15 ± | 0.19^{a} | 1.17 ± | 0.14^{a} | 0.38 ± | 0.04^{b} |
| Epididymal fat | 3.04 ± | 0.21 | 8.28 ± | 0.61^{a} | 7.92 ± | 0.60^{c} | 8.42 ± | 0.88^{a} | 8.50 ± | 0.71^{a} | 3.67 ± | 0.45^{c} |
| Retroperitoneum fat | 0.88 ± | 0.07 | 2.32 ± | 0.08***^{a} | 2.21 ± | 0.15^{a} | 2.01 ± | 0.22^{b} | 2.19 ± | 0.22^{a} | 1.34 ± | 0.32^{b} |
| Subcutaneous fat | 1.61 ± | .13 | 4.95 ± | 0.41***^{ab} | 4.88 ± | 0.15^{ab} | 5.47 ± | 0.47^{a} | 3.78 ± | 0.84^{a} | 1.99 ± | 0.19^{c} |
| Mesentery fat | 1.16 ± | 0.06 | 2.95 ± | 0.23***^{a} | 2.43 ± | 0.26^{a} | 2.62 ± | 0.44^{a} | 2.93 ± | 0.34^{a} | 0.87 ± | 0.09^{b} |
| Visceral fat | 7.13 ± | 0.48 | 19.74 ± | 0.28***^{a} | 18.36 ± | 0.54^{a} | 19.67 ± | 0.41^{a} | 18.22 ± | 0.42^{c} | 8.26 ± | 0.17^{a} |
| Interscapular WAT | 1.54 ± | 0.10 | 4.42 ± | 0.04***^{a} | 4.56 ± | 0.04^{a} | 4.54 ± | 0.05^{a} | 4.43 ± | 0.05^{c} | 1.70 ± | 0.02^{a} |
| Interscapular BAT | 0.34 ± | 0.02 | 0.75 ± | 1.09***^{a} | 0.57 ± | 1.66^{ab} | 0.71 ± | 1.52^{a} | 0.73 ± | 1.98^{c} | 0.34 ± | 0.98^{a} |
| Total WAT | 9.01 ± | 0.59 | 24.16 ± | 1.34***^{a} | 22.93 ± | 2.20^{a} | 24.21 ± | 1.83^{a} | 22.65 ± | 2.37^{c} | 9.95 ± | 1.15^{a} |
| InitialBodyweight (g) | 19.20 ± | 0.37 | 18.97 ± | 0.36 | 19.00 ± | 0.54 | 19.45 ± | 0.42 | 19.08 ± | 0.59 | 18.99 ± | 0.35 |
| FinalBodyweight (g) | 29.37 ± | 0.58 | 39.38 ± | 1.40***^{a} | 40.22 ± | 1.36^{a} | 41.77 ± | 1.31^{a} | 41.26 ± | 1.93^{a} | 30.13 ± | 0.69^{b} |
| BWG (g/16 weeks) | 0.64 ± | 0.002 | 1.28 ± | 0.08***^{a} | 1.32 ± | 0.10^{a} | 1.39 ± | 0.07^{a} | 1.39 ± | 0.09^{a}* | 0.70 ± | 0.04^{b} |
| Food Intake (g/day) | 3.12 ± | 0.10 | 3.03 ± | 0.25 | 3.17 ± | 0.26 | 3.03 ± | 0.25 | 3.03 ± | 0.25 | 3.17 ± | 0.26 |
| Energy Intake (kcal/day) | 12.16 ± | 0.39 | 13.88 ± | 1.15 | 13.88 ± | 1.15 | 13.88 ± | 1.15 | 13.88 ± | 1.15 | 13.88 ± | 1.15 |
| FER | 0.029 ± | 0.001 | 0.060 ± | 0.002***^{a} | 0.059 ± | 0.003^{a} | 0.065 ± | 0.003^{a} | 0.065 ± | 0.003^{a} | 0.031 ± | 0.002^{b} |

Organ weights (g/100 g Bodyweight)

| | | | | | | | | | | | | |
|---|---|---|---|---|---|---|---|---|---|---|---|---|
| Muscle | 1.08 ± | 0.02 | 0.85 ± | 0.03***^{b} | 0.77 ± | 0.03^{b} | 0.82 ± | 0.03^{b} | 0.83 ± | 0.04^{b} | 1.01 ± | 0.02^{a} |
| Liver | 3.41 ± | 0.05 | 5.49 ± | 0.32***^{a} | 5.37 ± | 0.25^{a} | 5.22 ± | 0.15^{a} | 5.68 ± | 0.35^{a} | 4.35 ± | 0.08^{b} |
| Kidney | 0.98 ± | 0.02 | 0.81 ± | 0.04*^{b} | 0.77 ± | 0.04^{b} | 0.74 ± | 0.02^{b} | 0.84 ± | 0.05^{b} | 1.03 ± | 0.02^{a} |

Adipose times weight (g/100 g Body weight)

| | | | | | | | | | | | | |
|---|---|---|---|---|---|---|---|---|---|---|---|---|
| Perinephric fat | 0.43 ± | 0.04 | 1.25 ± | 0.09***^{a} | 0.92 ± | 0.10^{c} | 1.15 ± | 0.19^{a} | 1.17 ± | 0.14^{a} | 0.38 ± | 0.04^{b} |
| Epididymal fat | 3.04 ± | 0.21 | 8.28 ± | 0.61^{a} | 7.92 ± | 0.60^{c} | 8.42 ± | 0.88^{a} | 8.50 ± | 0.71^{a} | 3.67 ± | 0.45^{c} |
| Retroperitoneum fat | 0.88 ± | 0.07 | 2.32 ± | 0.08***^{a} | 2.21 ± | 0.15^{a} | 2.01 ± | 0.22^{b} | 2.19 ± | 0.22^{a} | 1.34 ± | 0.32^{b} |
| Subcutaneous fat | 1.61 ± | .13 | 4.95 ± | 0.41***^{ab} | 4.88 ± | 0.15^{ab} | 5.47 ± | 0.47^{a} | 3.78 ± | 0.84^{a} | 1.99 ± | 0.19^{c} |
| Mesentery fat | 1.16 ± | 0.06 | 2.95 ± | 0.23***^{a} | 2.43 ± | 0.26^{a} | 2.62 ± | 0.44^{a} | 2.93 ± | 0.34^{a} | 0.87 ± | 0.09^{b} |
| Visceral fat | 7.13 ± | 0.48 | 19.74 ± | 0.28***^{a} | 18.36 ± | 0.54^{a} | 19.67 ± | 0.41^{a} | 18.22 ± | 0.42^{c} | 8.26 ± | 0.17^{a} |
| Interscapular WAT | 1.54 ± | 0.10 | 4.42 ± | 0.04***^{a} | 4.56 ± | 0.04^{a} | 4.54 ± | 0.05^{a} | 4.43 ± | 0.05^{c} | 1.70 ± | 0.02^{a} |
| Interscapular BAT | 0.34 ± | 0.02 | 0.75 ± | 1.09***^{a} | 0.57 ± | 1.66^{ab} | 0.71 ± | 1.52^{a} | 0.73 ± | 1.98^{c} | 0.34 ± | 0.98^{a} |
| Total WAT | 9.01 ± | 0.59 | 24.16 ± | 1.34***^{a} | 22.93 ± | 2.20^{a} | 24.21 ± | 1.83^{a} | 22.65 ± | 2.37^{c} | 9.95 ± | 1.15^{a} |

Statistical significance between ND and NFD groups: *p < 0.05, **p < 0.01, ***p < 0.001.
Statistical significance among HFD, ERY, GLU, FRU, and PSI groups (p < 0.05); Mean^{a,b,c}.
BWG, weight gain: FER, food efficiency ratio = Weight gain/diet intake, diet efficiency

As shown in FIG. 1 and Table 2, the initial body weights of the mice in all experimental groups were almost the same but the body weights of the high-fat diet-induced obese mice increased significantly compared to those of mice in the normal diet group (ND) from 4 weeks after feeding. However, increases in the body weight of the PSI-fed obese mice were considerably inhibited from 4 weeks after diet feeding, and a result, their body weights were maintained at almost the same levels as those of the mice in the ND group. That is, the diet efficiency of the PSI group was significantly lower than those of other high-fat diet groups (HFD, ERY, GLU, and FRU) and was maintained at almost the same level as that of the ND group.

In addition, an investigation was made as to whether the body weight loss of the PSI group was caused by the reduced organ weights. To this end, the weights of the organs (muscles, livers, and kidneys) and adipose tissues (perinephric fat, epididymal fat, retroperitoneal fat, subcutaneous fat, mesenteric fat, visceral fat, interscapular WAT, interscapular BAT, and total WAT) were measured. As shown in Table 2, the weights of the muscles and kidneys per unit body weight of the mouse in the high-fat diet groups (HFD, ERY, GLU, and FRU) except the PSI group decreased significantly compared to those in the ND group and the weights of the livers per unit body weight of the mouse in the high-fat diet groups increased significantly compared to those in the ND group. However, the weights of the muscles and kidneys per unit body weight of the mouse in the PSI group were found to be similar to those in the ND group. The weights of perinephric fat, epididymal fat, retroperitoneal fat, subcutaneous fat, mesenteric fat, visceral fat, interscapular WAT, interscapular BAT, and total WAT per unit body weight of the mouse in the high-fat diet groups (HFD, ERY, GLU, and FRU) except the PSI group increased significantly whereas the weights of all kinds of fats in the PSI group decreased significantly and were maintained at almost the same levels as those in the ND group.

From these results, it can be seen that D-psicose inhibited weight gain in the high-fat diet-induced obese mice and reduced the diet efficiencies and the weights of the livers and adipose tissues per unit body weight of the mouse in the high-fat diet-induced obese mice to levels similar to those of the ND group. In conclusion, D-psicose is effective in normalizing body weight and body fat mass.

### Example 2: Influences of D-Psicose on Plasma Lipid Profiles of High-Fat Diet-Induced Obese Mice

The following experiment was conducted to investigate the influences of D-psicose on the plasma lipid profiles of high-fat diet-induced obese mice.

Plasma free fatty acid, phospholipid, apolipoprotein A-I (Apo A-I), and apolipoprotein B (ApoB B) levels were measured using Nittobo enzymatic kits (Nittobo medical Co., Tokyo, Japan). Plasma HDL-cholesterol, triglyceride (TG), and total cholesterol (total-C) levels were measured using Asan enzymatic kits (Asan, Seoul, South Korea).

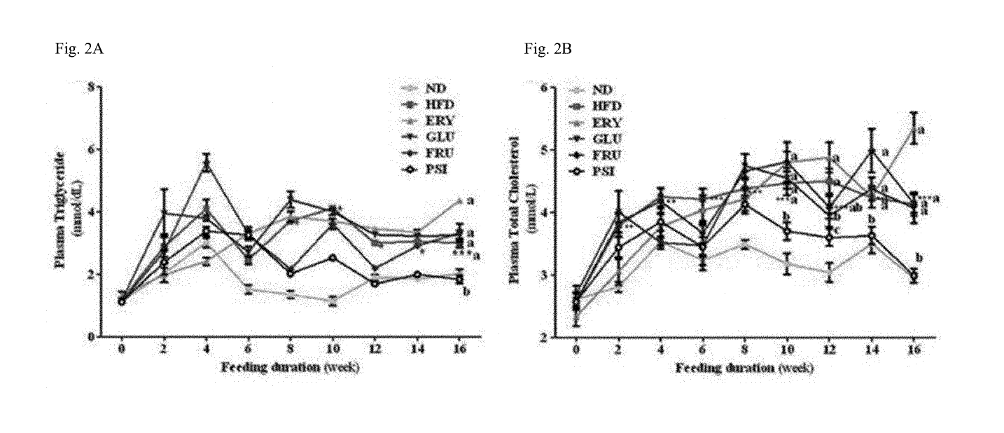

The results are shown in FIGS. 2A and 2B and Table 3.

TABLE 3

| | ND | | HFD | | ERY | | GLU | | FRU | | PSI | |
|---|---|---|---|---|---|---|---|---|---|---|---|---|
| FFA | 0.38 ± | 0.04 | 0.52 ± | 0.05*^{a} | 0.53 ± | 0.02^{a} | 0.49 ± | 0.06^{a} | 0.48 ± | 0.04^{a} | 0.41 ± | 0.02^{b} |
| (mmol/L) | | | | | | | | | | | | |
| TG | 0.84 ± | 0.06 | 1.28 ± | 0.06***^{a} | 1.25 ± | 0.07^{a} | 1.28 ± | 0.08^{a} | 1.17 ± | 0.03^{a} | 0.99 ± | 0.04^{b} |
| (mmol/L) | | | | | | | | | | | | |
| PL | 88.64 ± | 8.51 | 115.58 ± | 5.28**^{b} | 135.46 ± | 6.28^{a} | 104.86 ± | 6.92^{b} | 115.27 ± | 5.85^{b} | 107.23 ± | 2.82^{b} |
| (mg/dL) | | | | | | | | | | | | |
| Total-C | 2.95 ± | 0.17 | 4.17 ± | 0.17***^{b} | 5.35 ± | 0.98^{a} | 4.07 ± | 0.17^{b} | 4.14 ± | 0.29^{b} | 2.99 ± | 0.09^{c} |
| (mmol/L) | | | | | | | | | | | | |
| HDL-C | 0.91 ± | 0.04 | 1.16 ± | 0.06*^{a} | 1.22 ± | .08^{a} | 0.82 ± | 0.03^{b} | 0.83 ± | 0.09^{b} | 1.28 ± | .07^{a} |
| (mmol/L) | | | | | | | | | | | | |
| nonHDL-C | 2.01 ± | 0.15 | 3.00 ± | 0.15***^{b} | 4.36 ± | 0.73^{a} | 3.25 ± | 0.15^{b} | 3.32 ± | 0.29^{b} | 1.84 ± | .13^{c} |
| (mmol/L) | | | | | | | | | | | | |
| ApoA-I | 26.41 ± | 1.01 | 28.62 ± | 0.85*^{b} | 27.28 ± | 0.58^{b} | 27.23 ± | .95^{a} | 27.69 ± | 0.48^{b} | 29.39 ± | 0.95^{a} |
| (mg/dL) | | | | | | | | | | | | |
| ApoB | 7.35 ± | 0.89 | 9.16 ± | 0.4*^{ab} | 8.61 ± | 0.90^{b} | 9.32 ± | 1.04^{ab} | 9.96 ± | 0.79^{a} | 7.63 ± | 0.52^{c} |
| (mg/dL) | | | | | | | | | | | | |
| AI | 2.25 ± | 0.15 | 2.65 ± | 0.16^{ab} | 3.55 ± | 0.13^{ab} | 3.76 ± | 0.15^{ab} | 4.55 ± | 0.66^{a} | 1.42 ± | 0.13^{b} |
| HTR | 31.20 ± | 1.37 | 28.12 ± | 1.18^{b} | 23.95 ± | 3.53^{b} | 20.19 ± | 0.60^{b} | 20.62 ± | 2.56^{b} | 42.98 ± | 2.62^{a} |
| APO-A I/ | 3.59 ± | 0.56 | 3.12 ± | 0.17^{ab} | 3.16 ± | 0.34^{b} | 2.92 ± | 0.35^{b} | 2.77 ± | 0.21^{a} | 3.80 ± | 0.23^{a} |
| APO-B | | | | | | | | | | | | |

Statistical significance between ND and HFD groups: *p < 0.05, **p < 0.01, ***p < 0.001.
Statistical significance among HFD, ERY, GLU, FRU, and PSI groups (p < 0.05); Mean^{a,b,c}.
TG, Triglyceride; C, cholesterol; PL, phospholipid; HDL-C, high density lipoprotein cholesterol; Apo A-I, Apolipoprotein A-l; Apo-B, Apolipoprotein B; AI, atherogenic index, [(Total C) − HDL-C)]/HDL-C; HTR, (HDL-C/Total-C) × 100

As shown in FIGS. 2A and 2B and Table 3, the plasma free fatty acid, triglycerides, phospholipid, total-cholesterol, HDL cholesterol, non-HDL cholesterol, apolipoprotein A-I (Apo A-I), and apolipoprotein B (ApoB B) levels in the high-fat diet groups (HFD, ERY, GLU, and FRU) except the PSI group increased significantly compared to those in the ND group but the plasma free fatty acid, triglyceride, total cholesterol, non-HDL cholesterol, and Apo B levels in the PSI group were found to be similar to those in the normal diet group. Particularly, the HDL-cholesterol and Apo A-I levels in the PSI group were higher than those in the ND group and the arteriosclerosis index (AI) of the PSI group was found to be lower than that of the ND group.

From these results, it can be seen that D-psicose reduced the plasma free fatty acid, triglyceride, total cholesterol, non-HDL cholesterol, and Apo B levels in the high-fat diet-induced obese mice to values similar to those in the ND group, thus being effective in normalizing plasma lipid profiles. In addition, D-psicose increased the plasma HDL-cholesterol and Apo A-I levels in the high-fat diet-induced obese mice to higher values than those in the ND group and reduced the arteriosclerosis indices of the high-fat diet-induced obese mice to lower values than those in the ND group. Therefore, it is expected that D-psicose will be used to prevent arteriosclerosis.

### Example 3: Influences of D-Psicose on Plasma Leptin, Resistin, and Adiponectin Levels and Leptin:Adiponectin Ratio (L:A Ratio) in High-Fat Diet-Induced Obese Mice

The following experiment was conducted to investigate the influences of D-psicose on plasma leptin, resistin, and adiponectin levels and leptin:adiponectin ratio (L:A ratio) in high-fat diet-induced obese mice.

Plasma leptin, resistin, and adiponectin levels were measured using Bio-Rad multiplex kits (Hercules, Calif., USA). All samples were assayed in duplicate and analyzed using a Luminex 200 labmap system (Luminex, Austin, Tex., USA). Data analysis was performed using Bio-Plex Manager software version 4.1.1 (Bio-Rad, Hercules, Calif., USA).

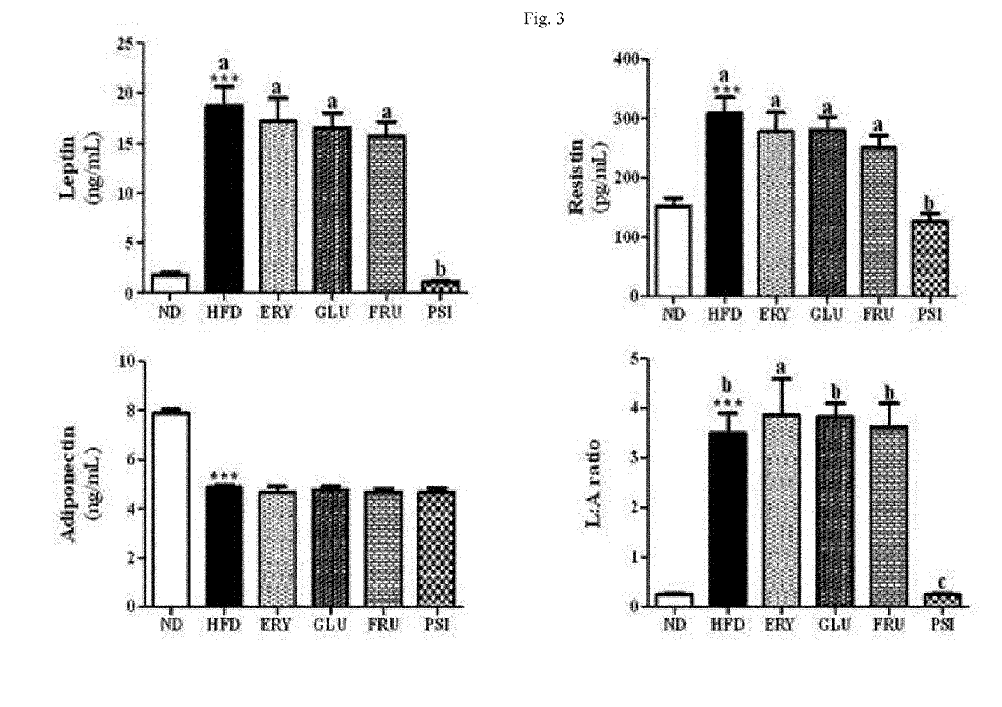

The results are shown in FIG. 3.

As shown in FIG. 3, the plasma leptin and resistin levels and the leptin:adiponectin ratios in the high-fat diet groups (HFD, ERY, GLU, and FRU) except the PSI group increased significantly compared to those in the ND group but the plasma leptin and resistin levels and the leptin:adiponectin ratio in the PSI group were reduced considerably to levels similar to those in the ND group.

From these results, it can be seen that D-psicose reduced the plasma leptin and resistin levels and the leptin:adiponectin ratios in the high-fat diet-induced obese mice to normal values.

### Example 4: Influences of D-Psicose on Hepatic Lipid Profiles, Hepatic Lipid Regulating Enzyme Activities, and Hepatic Tissue Morphologies of High-Fat Diet-Induced Obese Mice

The following experiment was conducted to investigate the influences of D-psicose on hepatic lipid profiles, hepatic lipid regulating enzyme activities, and hepatic tissue morphologies of high-fat diet-induced obese mice.

### Example 4-1. Hepatic Lipid Profiles

Hepatic lipids were extracted from mice in the normal diet group (ND) and high-fat diet groups (HFD, ERY, GLU, FRU, and PSI) and dried. Then, each of the dried hepatic lipid extracts was dissolved in 1 ml of ethanol. 200 μl of the lipid solution was emulsified in a solution of Triton X-100 and sodium cholate in distilled water. Hepatic fatty acid, triglyceride, and cholesterol levels were analyzed using the same enzymatic kits as those used in Example 2.

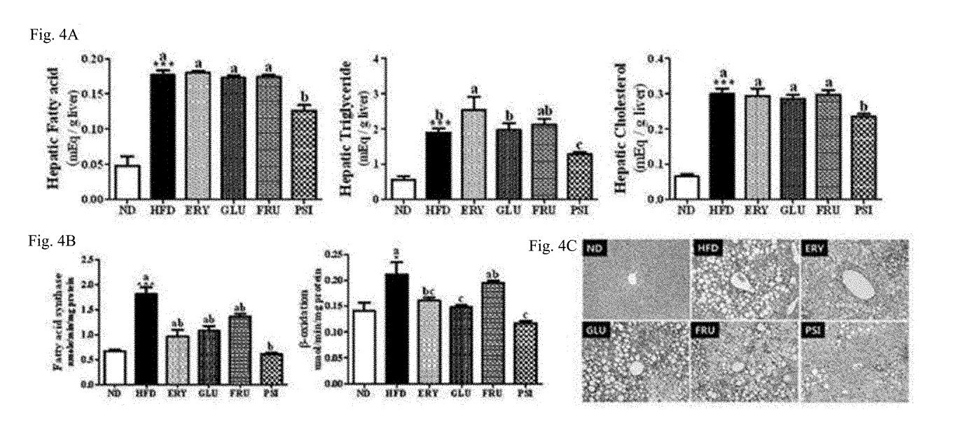

The results are shown in FIG. 4A.

As shown in FIG. 4A, the hepatic fatty acid, triglyceride, and cholesterol levels in the high-fat diet groups (HFD, ERY, GLU, FRU, and PSI) were found to be significantly higher than those in the ND group but the hepatic fatty acid, triglyceride, and cholesterol levels in the PSI group decreased significantly compared to those in other high-fat diet groups (HFD, ERY, GLU, and FRU).

### Example 4-2. Hepatic Lipid Regulating Enzyme Activity

Samples were prepared and analyzed according to the method developed by Hulcher and Oleson. Specifically, the activity of fatty acid synthase (FAS) as a hepatic lipid regulating enzyme was measured by spectrophotometric assay according to the method described by Nepokroeff et al. Each sample was mixed with 100 μl of cytoplasmic fraction and the mixture was allowed to react at 30° C. for 2 min. A reduction in absorbance at 340 nm was measured. Fatty acid synthase (FAS) activity units were expressed as nanomoles (nmol) of NADPH oxidized for 1 min per mg of cytoplasmic fraction. Fatty acid β-oxidation activity was measured by monitoring the reduction of NAD⁺ to NADH in the presence of palmitoyl-CoA, as described by Lazarow. β-oxidation activity units were expressed as nanomoles (nmol) of NADH produced for 1 min per mg of mitochondrial protein.

The results are shown in FIG. 4B.

As shown in FIG. 4B, the FAS activities and fatty acid β-oxidation activities in the high-fat diet groups (HFD, ERY, GLU, and FRU) except the PSI group increased significantly compared to those in the ND group but the FAS activities and fatty acid β-oxidation activities in the PSI group decreased significantly compared to those in other high-fat diet groups and were found to be similar to those in the ND group.

### Example 4-3. Hepatic Tissue Morphologies

Liver tissues were removed from the mice in the normal diet group (ND) and high-fat diet groups (HFD, ERY, GLU, FRU, and PSI) and fixed in a buffer solution of 10% formalin. The fixed liver tissues were embedded in paraffin. 4-mm sections were prepared from the liver tissues and their cross-sections were dyed with hematoxylin and eosin. Stained areas were observed using an optical microscope at a magnification of 200× (Nikon, Tokyo, Japan).

The results are shown in FIG. 4C.

As shown in FIG. 4C, the accumulation of adipocytes in the liver tissues of the high-fat diet groups (HFD, ERY, GLU, and FRU) except the PSI group was more distinctly observed than in the liver tissues of the ND group and the size of adipocytes in the liver tissues of the PSI group was smaller than that in the liver tissues of other high-fat diet groups.

These results reveal that D-psicose decreased the levels of fatty acids, triglycerides, and cholesterol, FAS activities, and adipocyte sizes in the livers of the high-fat diet-induced obese mice. In conclusion, D-psicose is effective in inhibiting fatty liver. In addition, D-psicose reduced hepatic fatty acid β-oxidation activities, which had been increased by high-fat diets, to levels similar to those in the normal diet group. In conclusion, D-psicose is effective in maintaining the homeostasis of hepatic lipid metabolism at the normal level.

### Example 5: Influences of D-Psicose on Lipid Regulating Enzyme Activities and Tissue Morphologies in Adipose Tissues of High-Fat Diet-Induced Obese Mice

The following experiment was conducted to investigate the influences of D-psicose on lipid regulating enzyme activities and tissue morphologies in the adipose tissues of high-fat diet-induced obese mice.

### Example 5-1. Lipid Regulating Enzyme Activities in Adipose Tissues

Samples were prepared and analyzed according to the method developed by Hulcher and Oleson. Specifically, the activity of fatty acid synthase (FAS) as a lipid regulating enzyme of epididymal white adipose tissue was measured by spectrophotometric assay according to the method described by Nepokroeff et al. Each sample was mixed with 100 μl of cytoplasmic fraction and the mixture was allowed to react at 30° C. for 2 min. A reduction in absorbance at 340 nm was measured. FAS activity units were expressed as nanomoles (nmol) of NADPH oxidized for 1 min per mg of cytoplasmic fraction. Fatty acid β-oxidation activity was measured by monitoring the reduction of NAD⁺ to NADH in the presence of palmitoyl-CoA, as described by Lazarow. β-oxidation activity units were expressed as nanomoles (nmol) of NADH produced for 1 min per mg of mitochondrial protein.

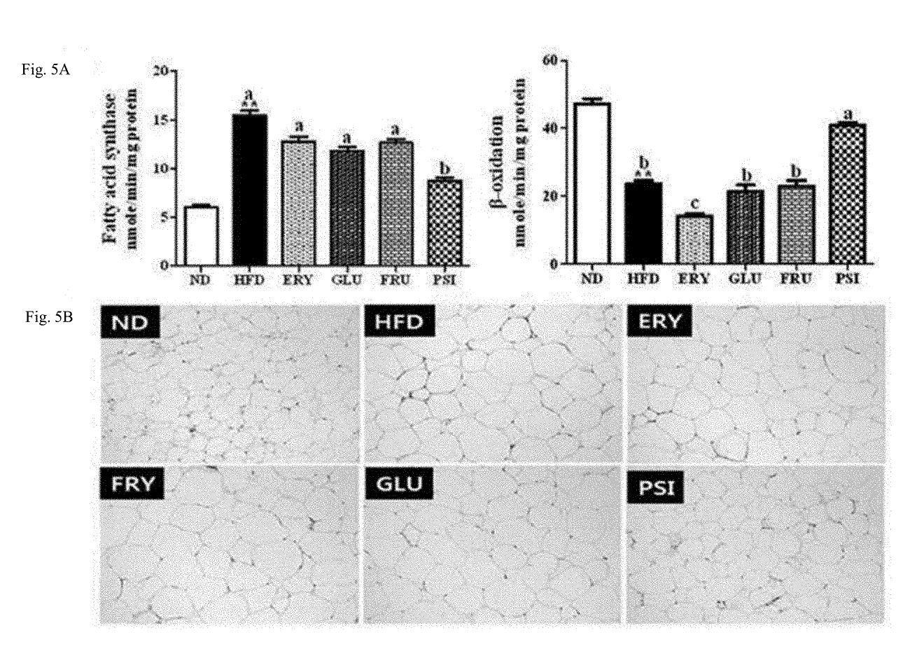

The results are shown in FIG. 5A.

As shown in FIG. 5A, the FAS activities in the high-fat diet groups (HFD, ERY, GLU, and FRU) except the PSI group increased significantly compared to those in the ND group and the fatty acid β-oxidation activities in the high-fat diet groups (HFD, ERY, GLU, and FRU) except the PSI group decreased significantly compared to those in the ND group. The FAS activities in the PSI group decreased significantly compared to those in other high-fat diet groups whereas the fatty acid β-oxidation activities in the PSI group increased significantly compared to those in other high-fat diet groups and were found to be similar to those in the normal diet group. From these results, it can be seen that D-psicose reduced the synthesis of fatty acids and increased the oxidation of fatty acids in the adipose tissues of high-fat diet-induced obese mice. In conclusion, D-psicose is effective in reducing body fat mass.

### Example 5-2. Adipose Tissue Morphologies

Epididymal WATs were removed from the mice in the normal diet group (ND) and high-fat diet groups (HFD, ERY, GLU, FRU, and PSI) and fixed in a buffer solution of 10% formalin. The fixed epididymal WATs were embedded in paraffin. 4-mm sections were prepared from the epididymal WATs and their cross-sections were dyed with hematoxylin and eosin. Stained areas were observed using an optical microscope at a magnification of 200× (Nikon, Tokyo, Japan).

The results are shown in FIG. 5B.

As shown in FIG. 5B, an increase in the size of adipocytes in the epididymal WATs of the high-fat diet groups (HFD, ERY, GLU, and FRU) except the PSI group was distinctly observed compared to in the epididymal WATs of the ND group. The size of adipocytes in the PSI group was found to be relatively small compared to that in the other high-fat diet groups.

From these results, it can be seen that D-psicose reduced the synthesis of fatty acids and increased the oxidation of fatty acids in the adipose tissues of high-fat diet-induced obese mice, resulting in a reduction in the size of adipocytes and an inhibition of lipid accumulation. In conclusion, D-psicose is effective in normalizing body fat mass to the normal level.

### Example 6: Influences of D-Psicose on mRNA Expression of Genes Involved in Fatty Acid Synthesis and Oxidation in the Livers of High-Fat Diet-Induced Obese Mice

The following experiment was conducted to investigate the influences of D-psicose on mRNA expression of genes (FAS, ACC1, CPT1α, and CPT2) involved in fatty acid synthesis and oxidation in the livers of high-fat diet-induced obese mice.

Samples were prepared and analyzed as previously described. Specifically, total RNA was synthesized into cDNA using a QuantiTect Reverse Transcription kit (QIAGEN Gmb1h, Hilden, Germany). RNA expression was quantified with real-time quantitative PCR using a QuantiTect SYBR Green PCR kit (QIAGEN Gmb1h, Hilden, Germany). Primers were designed to detect FAS (fatty acid synthase, 14101), ACC1 (Acetyl-CoA carboxylase 1, 107476), CPT1α (Carnitine palmitoyltransferase 1α, 12894), and CPT2 (Carnitine palmitoyltransferase 2, 12896). GAPDH was used as an internal transcription marker. The reaction was performed a total of 40 cycles (each consisting of 15 sec at 94° C., 30 sec at 58° C., 30 sec at 72° C., and 15 sec at 65° C.). Fluorescence signals were monitored every cycle and the resulting threshold cycles (Ct) were analyzed. mRNA expression in each experimental group was quantified using a CFX96 Real time system (Bio-rad, USA).

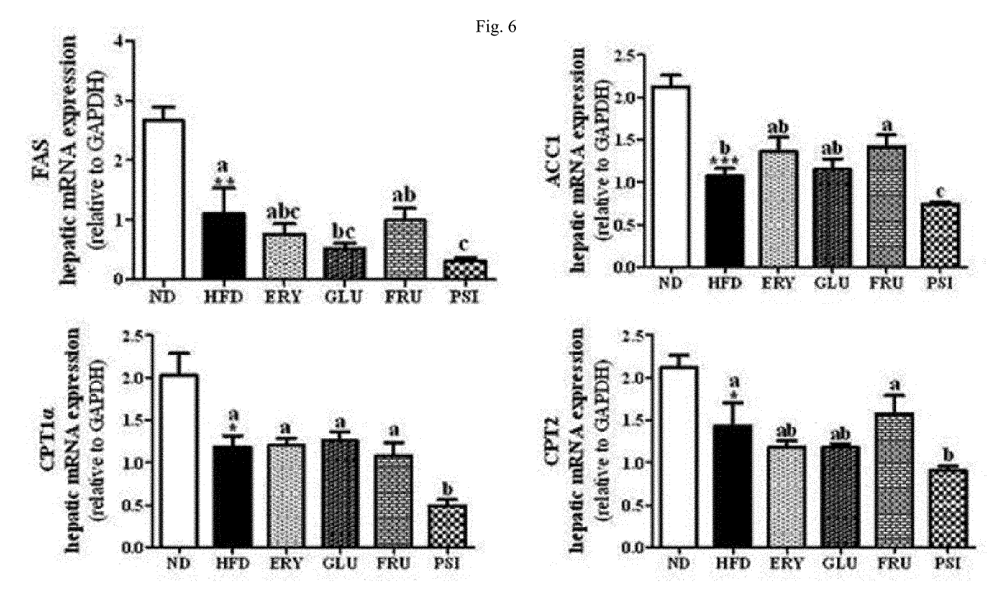

The results are shown in FIG. 6.

As shown in FIG. 6, mRNA expression levels of genes (FAS and ACC1) involved in hepatic fatty acid synthesis and genes (CPT1α and CPT2) involved in fatty acid oxidation in all high-fat diet groups (HFD, ERY, GLU, FRU, PSI) were significantly lower than those in the ND group. Particularly, mRNA expression levels of genes (FAS and ACC1) involved in hepatic fatty acid synthesis and genes (CPT1α and CPT2) involved in fatty acid oxidation in the PSI group were much significantly lower than those in the ND group.

From these results, it can be seen that D-psicose reduced mRNA expression of genes involved in fatty acid synthesis in the livers of high-fat diet-induced obese mice to inhibit fat production in the livers.

### Example 7: Influences of D-Psicose on Lipid Excretion in Feces from High-Fat Diet-Induced Obese Mice

The following experiment was conducted to investigate the influences of D-psicose on lipid excretion in feces from high-fat diet-induced obese mice.

Lipids were extracted from feces from mice in the normal diet group (ND) and the high-fat diet groups (HFD, ERY, GLU, FRU, and PSI) and dried. Then, each of the dried lipid extracts was dissolved in 1 ml of ethanol. 200 μl of the lipid solution was emulsified in a solution of Triton X-100 and sodium cholate in distilled water. Triglyceride, cholesterol, and fatty acid levels in the feces were analyzed using the same enzymatic kits as those used in Example 2.

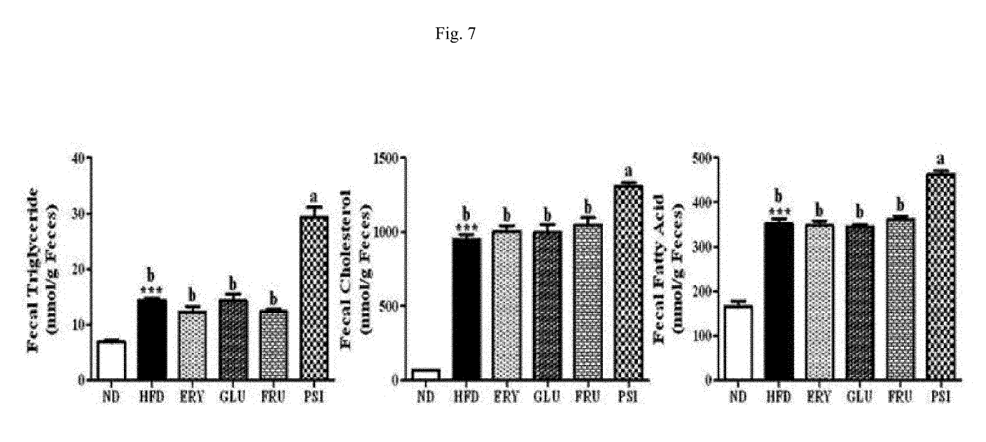

The results are shown in FIG. 7.

As shown in FIG. 7, the triglyceride, cholesterol, and fatty acid levels in the feces from the high-fat diet groups (HFD, ERY, GLU, FRU, and PSI) were found to be significantly higher than those from the normal diet group. Particularly, the triglyceride, cholesterol, and fatty acid levels in the feces from the PSI group were confirmed to be significantly higher than those from other high-fat diet groups (HFD, ERY, GLU, and FRU).

From these results, it can be seen that D-psicose increased lipid excretion in feces from high-fat diet-induced obese mice, which is associated with the inhibitory effect of D-psicose on enteric fat absorption.

### Example 8: Influences of D-Psicose on mRNA Expression of Genes Involved in Lipid Absorption in the Small Intestines of High-Fat Diet-Induced Obese Mice

The following experiment was conducted to investigate the influences of D-psicose on mRNA expression of genes (CD36, FATP4, and ApoB48) involved in lipid absorption and genes (ABCG5 and ABCG8) involved in excretion in the small intestines of high-fat diet-induced obese mice.

Samples were prepared and analyzed as previously described. Specifically, total RNA was synthesized into cDNA using a QuantiTect Reverse Transcription kit (QIAGEN Gmb1h, Hilden, Germany). RNA expression was quantified with real-time quantitative PCR using a QuantiTect SYBR Green PCR kit (QIAGEN Gmb1h, Hilden, Germany). Primers were designed to detect CD36 (cluster of differentiation 36, 12491), ApoB48 (apolipoprotein B 48, 238055), FATP4 (fatty acid transporter 4, 26569), ABCG5 (ATP-binding cassette sub-family G member5, 27409), and ABCG8 (ATP-binding cassette sub-family G member8, 67470). GAPDH was used as an internal transcription marker. The reaction was performed a total of 40 cycles (each consisting of 15 sec at 94° C., 30 sec at 58° C., 30 sec at 72° C., and 15 sec at 65° C.). Fluorescence signals were monitored every cycle and the resulting threshold cycles (Ct) were analyzed. mRNA expression in each experimental group was quantified using a CFX96 Real time system (Bio-rad, USA).

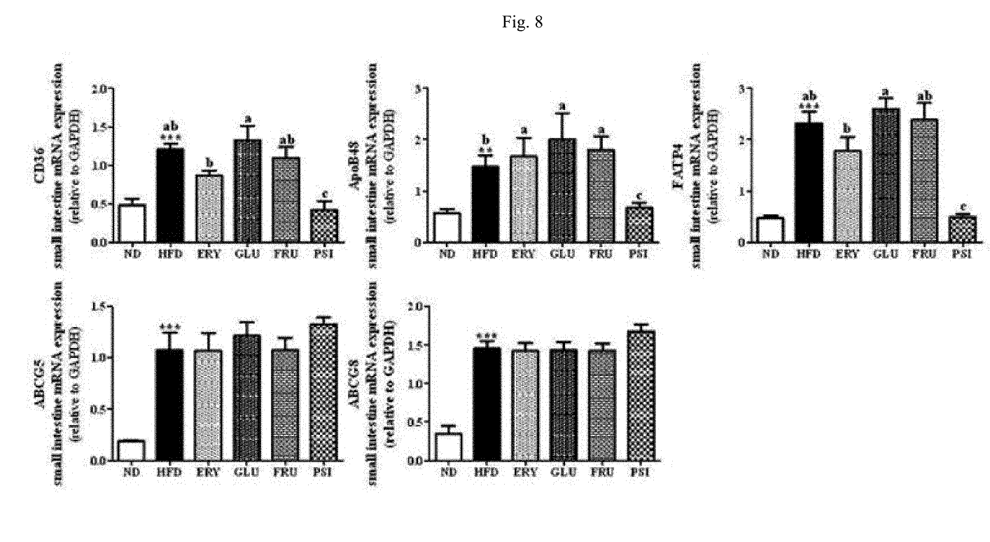

The results are shown in FIG. 8.

As shown in FIG. 8, mRNA expression levels of genes (CD36, FATP4, and ApoB48) involved in lipid absorption in the small intestine were significantly higher in the high-fat diet groups (HFD, ERY, GLU, and FRU) except the PSI group than those in the ND group. mRNA expression levels of genes (CD36, FATP4, and Apo B48) involved in lipid absorption in the small intestine were significantly lower in the PSI group than those in other high-fat diet groups (HFD, ERY, GLU, and FRU) and were maintained at the same levels as those in the normal diet group.

From these results, it can be seen that D-psicose reduced mRNA expression of genes involved in lipid absorption in the small intestines of high-fat diet-induced obese mice. In conclusion, D-psicose has an inhibitory effect on lipid availability because of its ability to inhibit lipid absorption in the small intestine.

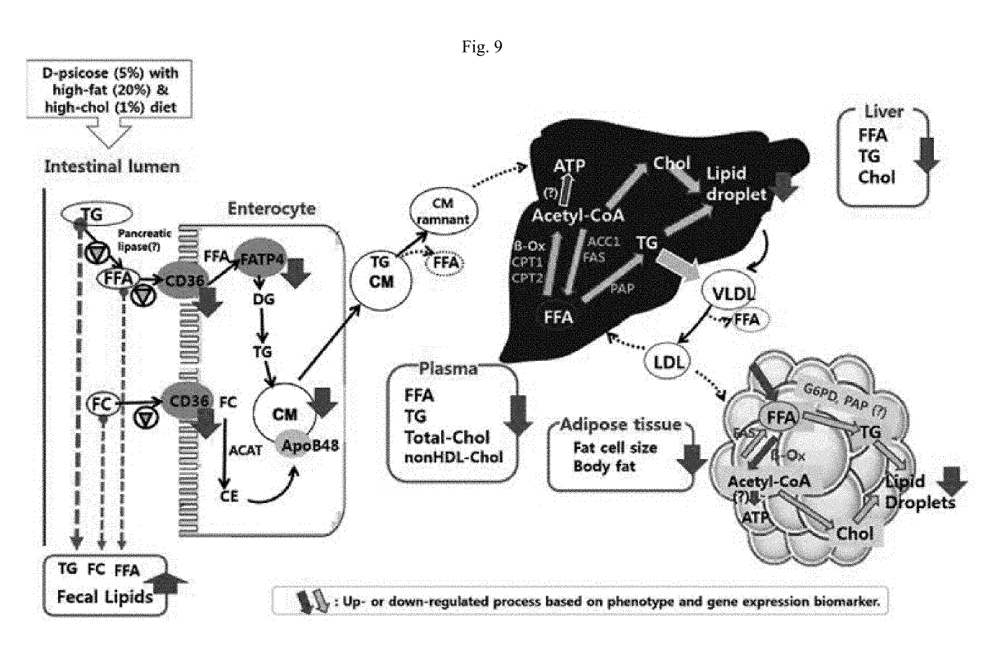

Based on the results obtained in Examples 1-8, the roles of D-psicose on lipid metabolism in the small intestines, hepatic tissues, and adipose tissues of high-fat diet-induced obese mice are briefly summarized in FIG. 9.

The composition according to the present invention was prepared into the following formulations.

### Preparation Example 1: Preparation of Pharmaceutical Formulations

### 1. Preparation of Powders

| | |
|---|---|
| D-psicose | 200 mg |
| Lactose | 100 mg |

The ingredients were mixed together and filled in air-tight bags to prepare powders

### 2. Preparation of Tablets

| | |
|---|---|
| D-psicose | 200 mg |
| Corn starch | 100 mg |
| Lactose | 100 mg |
| Magnesium stearate | 2 mg |

The ingredients were mixed together and compressed to prepare tablets according to a suitable method known in the art.

### 3. Preparation of Capsules

| | |
|---|---|
| D-psicose | 200 mg |
| Corn starch | 100 mg |
| Lactose | 100 mg |
| Magnesium stearate | 2 mg |

The ingredients were mixed together and filled in gelatin capsules to prepare capsules according to a suitable method known in the art.

### 4. Preparation of Injectables

| | |
|---|---|
| D-psicose | 200 mg |
| Mannitol | 100 mg |
| Na₂HPO₄•12H₂O | 2 mg |
| Sterilized distilled water for injection | q.s |

The ingredients were mixed together in ampules (2 ml each) to prepare injectables according to a suitable method known in the art.

### Preparation Example 2: Preparation of Foods

Foods including D-psicose were prepared by the following procedures:

### 1. Preparation of Cooking Sauces

Healthy cooking sauces including 20-95 wt % of D-psicose were prepared.

### 2. Preparation of Tomato Ketchups and Sauces

Healthy tomato ketchups and sources including 0.2-1.0 wt % of D-psicose were prepared.

### 3. Preparation of Flour Foods

0.5-5.0 wt % of D-psicose was added to flour. The mixture was used to prepare breads, cakes, cookies, crackers, and healthy flour products.

### 4. Preparations of Soups and Gravies

0.1-5.0 wt % of D-psicose was added to soups and gravies for healthy meat processed products and flour products.

### 5. Preparation of Ground Beef

Healthy ground beef including 10 wt % of D-psicose was prepared.

### 6. Preparation of Dairy Products

5-10 wt % of D-psicose was added to milk. The mixture was used to prepare dairy products such as butters and ice creams.

### Preparation Example 3: Preparation of Beverages

### 1. Preparation of Carbonated Beverages

10-15% of D-psicose, 5-10% of sugar, 0.05-0.3% of citric acid, 0.005-0.02% of caramel, and 0.1-1% of vitamin were mixed together. The mixture was mixed with 75-80% of purified water to prepare a syrup. The syrup was sterilized at 85-98° C. for 20-180 sec and mixed with cooling water in a ratio of 1:4. The mixture was injected with 0.5-0.82% of carbonic acid gas to prepare a carbonated beverage containing D-psicose.

### 2. Preparation of Healthy Beverages

D-psicose (solid content: 2.5%, 97.16%), jujube extract (65 brix, 2.67%), fruit-beverage complex extract (solid content: 70%, 0.12%), vitamin C (0.02%), calcium pantothenate (0.02%), and licorice extract (solid content: 65%, 0.01%) were mixed together. The mixture was homogenized, instantaneously sterilized, and packaged in small packaging containers such as glass bottles and PET bottles to prepare healthy beverages.

### 3. Preparation of Vegetable Juices

0.5 g of D-psicose was added to 1,000 ml of a tomato or carrot juice to prepare a healthy vegetable juice.

### 4. Preparation of Fruit Juices

0.1 g of D-psicose was added to an apple or grape juice to prepare a healthy fruit juice.

Although the present invention has been described herein with reference to the foregoing embodiments, it will be understood by those skilled in the art that the invention can be implemented in other specific forms without changing the spirit or essential features of the invention. Therefore, it should be noted that the forgoing embodiments are merely illustrative in all aspects and are not to be construed as limiting the invention.

### INDUSTRIAL APPLICABILITY

In the present invention, the physiological activity of D-psicose was clarified by assigning isocaloric diets to diet groups to exclude the effect of D-psicose on calorie reduction. As a result, it was found that D-psicose has functions of inhibiting lipid absorption in the small intestine and considerably increasing lipid levels in feces to inhibit fat production and reduces body weight, body fat mass, and plasma lipid levels such that body weight, body fat mass, and plasma lipid profiles are normalized in a short time. Due to these advantages, it is expected that D-psicose will be used to prevent and/or treat lipid-related metabolic diseases.

1. A composition for inhibiting the absorption of lipids, promoting the excretion of the ingested lipids, or both, comprising D-psicose.

2. A method for inhibiting an absorption of an ingested lipids in a subject, promoting an excretion of the ingested lipids in the subject, or both, comprising administering the composition of claim 1 to the subject the subject.

3. The method according to claim 2, wherein the D-psicose is administered to the subject in an amount of 10 to 50 parts by weight, relative to 100 parts by weight of the ingested lipids.

4. The method according to claim 2, wherein administering the composition to the subject includes inhibiting a small intestine of the subject from the absorption of the ingested lipids.

5. A method of reducing food lipid absorption, promoting food lipid excretion or both, by using the composition of claim 1.

6. A method for inhibiting an activity of fatty acid synthase in a subject, the method comprising administering the composition of claim 1 to a subject in need thereof.

7. A method for preventing, ameliorating or treating hyperlipidemia, arteriosclerosis, fatty liver, or a combination thereof, the method comprising administering a pharmaceutically effective amount of D-psicose to a subject in need thereof, by administering the composition of claim 1 to the subject.

8. A method of preventing or treating lipid-related metabolic disease, the method comprising administering the composition of claim 1 to a subject in need thereof.

9. (canceled)
